(19) United States
(12) Patent Application Publication
(10) Pub. No.: US 2015/0298113 A1
(43) Pub. Date: Oct. 22, 2015

(54) Novel Ruthenium Complexes, A Method Of Producing Them, And Their Use In Olefin Metathesis

(71) Applicant: INSTYTUT CHEMII ORGANICZNEJ PAN, Warszawa (PL)

(72) Inventors: Karol GRELA, Warszawa (PL); Rafal GAWIN, Warszawa (PL); Michal PIECZYKOLAN, Zamosc (PL)

(21) Appl. No.: 14/653,857

(22) PCT Filed: Mar. 25, 2013
(86) PCT No.: PCT/IB2013/052368

§ 371 Date: Jun. 19, 2015

(30) Foreign Application Priority Data
Dec. 28, 2012 (PL) P 402293

(51) Int. Cl. B01J 31/24; C07D 307/935; C07C 41/30; C07F 15/00

(52) U.S. Cl. CPC B01J 31/2404; C07F 15/0046; C07D 307/935; C07C 41/30; B01J 2231/543; B01J 2231/52; B01J 2531/821

(57) ABSTRACT

The subject of the present invention are novel, chiral non-racemic ruthenium complexes defined by the general Formula 1,

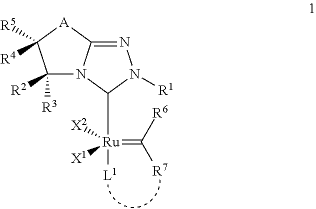

The present invention also relates to methods of manufacturing of a novel, chiral non-racemic ruthenium complexes defined by the general Formula 1 as well as their use in asymmetric olefin metathesis reactions.

The present invention relates to novel chiral, non-racemic complexes of ruthenium, for use as (pre)catalysts in the olefin metathesis reactions, a method of producing them and their use in the metathesis reactions of olefins (Chem. Rev. 2010, 110, 1746-1787).

The state of the art encompasses chiral, non-racemic carbene ruthenium complexes that act as (pre)catalysts, which make it possible to perform asymmetric metathesis reactions. These complexes posses NHC carbenes as a ligand that are that are derivatives of imidazolo-4,5-dihydro-2-ylidene (Chem. Soc. Rev. 2012, 41, 4389-4408). The synthesis of known chiral non-racemic ruthenium (pre)catalysts using metathesis reactions consists of many stages, and their precursors are difficult to obtain (J. Am. Chem. Soc. 2006, 128, 1840-1846; Organometallics 2007, 26, 2945-2949; J. Am. Chem. Soc. 2002, 124, 4954-4955).

Unexpectedly, it was shown that novel, chiral, non-racemic carbene ruthenium complexes defined by Formula 1,

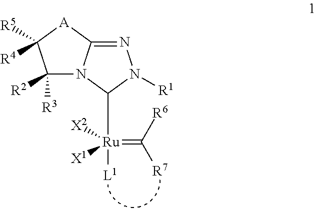

containing as a ligand chiral, non-racemic NHC carbenes that are derivatives of 1,2,4-triazol-5-ylidene make it possible to perform asymmetric metathesis reactions. The precursors of complex 1 are optically active amino-alcohols, which may be obtained from inexpensive amino-acids of natural origin. The synthesis of complex 1 consists of a lesser number of stages, in comparison to the syntheses of known chiral non-racemic ruthenium (pre)catalysts for metathesis reactions.

Complexes defined by Formula 1, according to the present invention are useful in a wide range of asymmetric metathesis reactions.

The subject of the present invention are novel, chiral non-racemic ruthenium complexes defined by the general Formula 1,

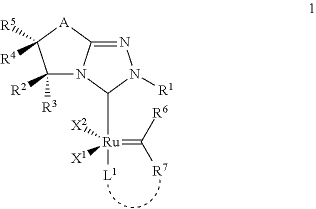

in which:
R¹ denotes a C₅-C₂₄ perfluoroaryl;
R², R³, R⁴ and R⁵ independently of one another denote a hydrogen atom, a halogen atom, a C₁-C₂₅ alkyl, a C₃-C₇ cycloalkyl, a C₁-C₂₅ alkoxyl, a C₅-C₂₄ aryloxyl, a C₅-C₂₀ heteroaryloxyl, a C₅-C₂₄ aryl, a C₅-C₂₀ heteroaryl, a C₇-C₂₄ aralkyl, a C₅-C₂₄ perfluoroaryl, or 3-12 membered heterocycle, wherein the groups R², R³, R⁴ and R⁵ may be mutually connected into a ring;
A denotes a —CH₂—, —O— or —OCH₂— group;
R⁶ and R⁷ independently of one another denote a hydrogen atom, a halogen atom, a C₁-C₂₅ alkyl, a C₁-C₂₅ perfluoroalkyl, a C₂-C₂₅ alkene, a C₃-C₇ cycloalkyl, a C₂-C₂₅ alkenyl, a C₃-C₂₅ cycloalkenyl, a C₂-C₂₅ alkynyl, a C₃-C₂₅ cycloalkynyl, a C₁-C₂₅ alkoxyl, a C₅-C₂₄ aryloxyl, a C₅-C₂₀ heteroaryloxyl, a C₅-C₂₄ aryl, a C₅-C₂₀ heteroaryl, a C₇-C₂₄ aralkyl, a C₅-C₂₄ perfluoroaryl, a 3-12 membered heterocycle wherein the alkyl groups may be mutually connected into a ring, wherein R⁶ and R⁷ preferably denote a hydrogen, an aryl substituted with a nitro (—NO₂), cyanide (—CN), carboxyl (—COOH), ester (—COOR′), amide (—CONR′₂), sulfonyl (—SO₂R′), formyl (—CHO), sulfonamide (—SO₂NR′₂), or ketone (—COR′) group, in which R′ has the following meaning: a C₁-C₅ alkyl, a C₁-C₅ perfluoroalkyl, a C₅-C₂₄ aryl, a C₇-C₂₄ aralkyl, a C₅-C₂₄ perfluoroaryl;
L¹ denotes a neutral ligand;
X¹ and X² denote an anionic ligand.

The neutral ligand L¹ is selected from among the groups encompassing P(R′)₃, P(OR′)₃, O(R′)₃, N(R′)₃, where each R′ independently denotes a C₁-C₁₂ alkyl, a C₃-C₁₂ cycloalkyl, a C₅-C₂₀ aryl, a C₇-C₂₄ aralkyl, a C₅-C₂₄ perfluoroaryl, or a 5-12 membered heteroaryl; The neutral ligand L¹ may also be a pyridine or substituted pyridine;

The anionic ligands X¹ and X² are independently selected from groups encompassing halide anions, as well as —CN, —SCN, —OR′, —SR′, —O(C═O)R′, —O(SO₂)R′, —OSi(R′)₃ groups, where R′ denotes a C₁-C₁₂ alkyl, a C₃-C₁₂ cycloalkyl, a C₂-C₁₂ alkenyl, or a C₅-C₂₀ aryl, which may possibly be substituted with at least one C₁-C₁₂ alkyl, a C₁-C₁₂ perfluoroalkyl, a C₁-C₁₂ alkoxyl, a C₅-C₂₄ aryloxyl, a C₅-C₂₀ heteroaryloxyl or a halogen atom.

In a preferable embodiment of the present invention, complex 1 has a structure defined by the general Formula 1a

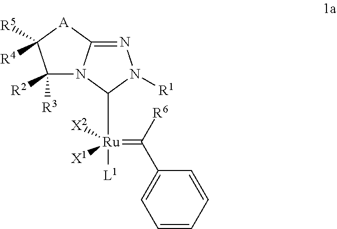

in which:
R¹, R², R³, R⁴, R⁵, A, L¹, X¹, and X² have the same meaning as in Formula 1
R⁶ denotes a hydrogen.

In another preferable embodiment, complex 1 has a structure defined by the general Formula 1b

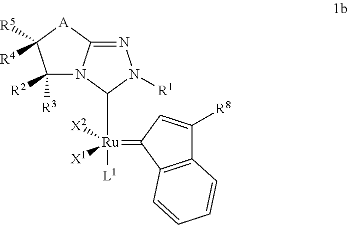

in which:
R¹, R², R³, R⁴, R⁵, A, L¹, X¹ and X² have the same meaning as in Formula 1
R⁸ denotes a hydrogen atom, a C₅-C₂₀ aryl, a C₅-C₂₀ heteroaryl, a C₇-C₂₄ aralkyl, vinyl or allenyl.

In another preferable embodiment, complex 1 has a structure defined by the general Formula 1c

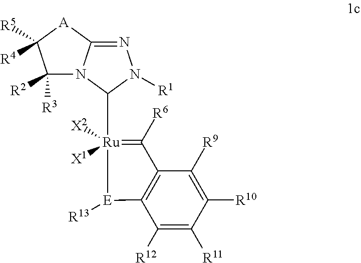

in which:
R¹, R², R³, R⁴, R⁵, A, L¹, X¹ and X² have the same meaning as in Formula 1 R⁶ denotes a hydrogen;
R₉, R₁₀, R¹¹, R¹² independently of one another denote a hydrogen atom, a halogen atom, a C₁-C₂₅ alkyl, a C₁-C₂₅ perfluoroalkyl, a C₂-C₂₅ alkene, a C₃-C₇ cycloalkyl, a C₂-C₂₅ alkenyl, a C₃-C₂₅ cycloalkenyl, a C₂-C₂₅ alkynyl, a C₃-C₂₅ cycloalkynyl, a C₅-C₂₄ aryl, a C₅-C₂₀ heteroaryl, a C₇-C₂₄ aralkyl, a C₅-C₂₄ perfluoroaryl, or 3-12 membered heterocycle wherein the alkyl groups may be mutually connected into a ring, an ether (−OR′), thioether (—SR′), nitro (—NO₂), cyanide (—CN), carboxyl (—COOH), ester (—COOR′), amide (—CONR′₂), imide (—CONR′COR′), amino (—NR′₂), amide (NR′COR′), sulfonamide (—NR′SO₂R′), sulfonyl (—SO₂R′), formyl (—CHO), sulfonamide (—SO₂NR′₂) or ketone (—COR′) group, in which R′ has the following meaning: a C₁-C₅ alkyl, a C₁-C₅ perfluoroalkyl, a C₅-C₂₄ aryl, a C₅-C₂₄ perfluoroaryl, a C₇-C₂₄ aralkyl, wherein the alkyl groups may be mutually connected into a ring; preferably, R⁹, R¹⁰, R¹¹, R¹² denotes a hydrogen;
R¹³ denotes a hydrogen atom, a C₁-C₂₅ alkyl, a C₁-C₂₅ perfluoroalkyl, a C₃-C₇ cycloalkyl, a C₅-C₂₄ aryl, a C₅-C₂₄ perfluoroaryl, a C₅-C₂₅ heteroaryl, a C₇-C₂₄ aralkyl, a 3-12 membered heterocycle wherein the alkyl groups may be mutually connected into a ring, an acyl —COR′, cyanide (—CN), carboxyl (—COOH), ester (—COOR′), amide (—CONR′₂), sulfonyl (—SO₂R′), formyl (—CHO), sulfonamide (—SO₂NR′₂), or ketone (—COR′) group, in which R′ has the following meaning: a C₁-C₅ alkyl, a C₁-C₅ perfluoroalkyl, a C₅-C₂₄ aryl, a C₅-C₂₄ perfluoroaryl, or a C₇-C₂₄ aralkyl;
E denotes an oxygen atom.

Preferably, a complex according to the present invention is characterised in that
R¹ denotes pentafluorophenyl;
R², R³, R⁴ and R⁵ independently of one another denote a hydrogen atom, a C₁-C₂₅ alkyl, a C₃-C₇ cycloalkyl, a C₅-C₂₄ aryl, a C₅-C₂₀ heteroaryl, a C₇-C₂₄ aralkyl, a C₅-C₂₄ perfluoroaryl, or a 3-12 membered heterocycle, wherein the groups R², R³, R⁴ and R⁵ may be mutually connected into a ring;
A denotes a —CH₂—, —O—, or —OCH₂— group;
L¹ denotes a neutral ligand selected from groups encompassing tricyclohexylphosphine, triphenylphosphine, pyridine, 3-bromopyridine;
X¹ and X² denote chlorine, bromine or iodine.

The subject of the present invention is also a method of producing complexes of ruthenium defined by the general Formula 1, which encompasses reactions of carbene complexes of ruthenium defined by Formula 2,

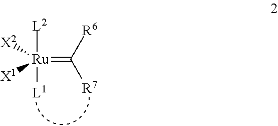

in which:
R⁶, R⁷, L¹, X¹ and X² have the same meaning as in Formula 1
L¹ and L² have the same meaning as L¹ in Formula 1
   with chiral non-racemic carbenes defined by the general Formula 3 or with chiral non-racemic complexes of silver defined by the general Formula 4 or with carbenes formed from precursors of chiral non-racemic carbenes defined by the general Formula 5 or 6,

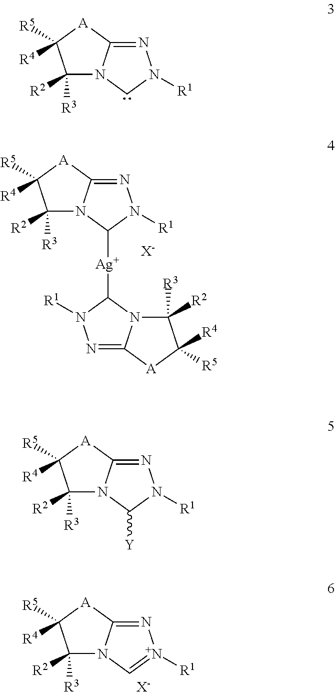

in which:
R¹, R², R³, R⁴, R⁵ and A have the same meaning as in Formula 1
X denotes a halide anion or BF₄⁻, PF₆₋, ClO₄⁻
Y denotes alkoxyl, pentafluorophenyl, —CCl₃.

In a preferable embodiment as a precursor of complex 1 use is made of complex 2 defined by the general Formula 2a

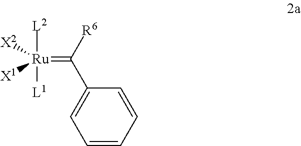

in which:
X¹ and X² have the same meaning as in Formula 1;
L¹ and L² have the same meaning as L¹ in Formula 1;
R⁶ denotes a hydrogen.

In another preferable embodiment as a precursor of complex 1 use is made of complex 2 defined by the general Formula 2b

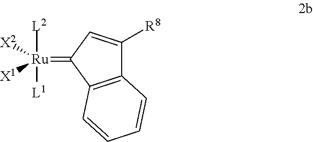

in which
X¹ and X² have the same meaning as in Formula 1;
L¹ and L² have the same meaning as L¹ in Formula 1;
R⁸ denotes a hydrogen atom, a C₅-C₂₀ aryl, a C₅-C₂₀ heteroaryl, a C₇-C₂₄ aralkyl, vinyl or allenyl.

In another preferable embodiment as a precursor of complex 1 use is made of complex 2 defined by the general Formula 2c

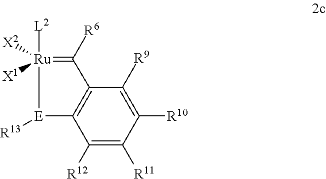

in which
X¹ and X² have the same meaning as in Formula 1;
L² has the same meaning as L¹ in Formula 1;
R⁶, R⁹, R¹⁰, R¹¹, R¹² and R¹³ have the same meaning as in Formula 1c;
E has the same meaning as in Formula 1c.

The production of complexes of ruthenium 1 from complexes of ruthenium 2 and carbenes 3 or with complexes of silver 4 or with precursor carbenes 5 is shown in Scheme I:

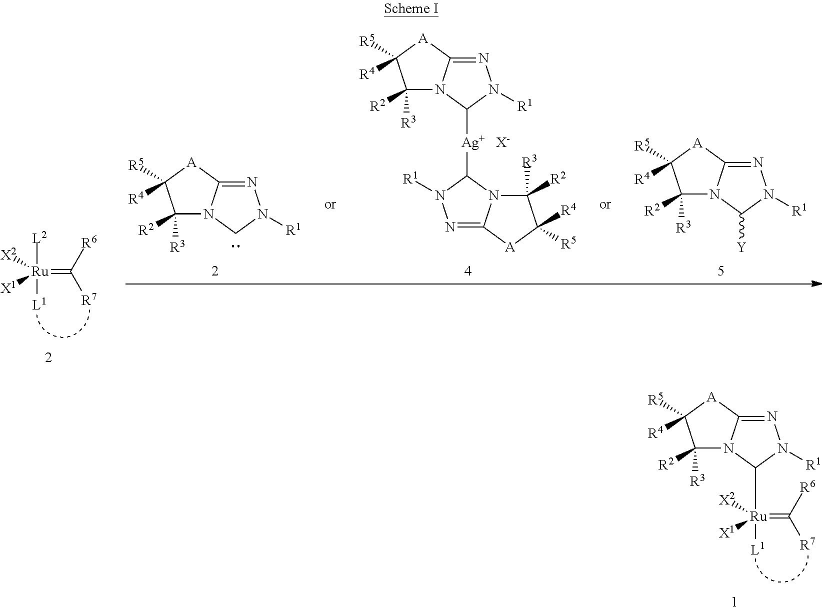

Preferably, reactions are conducted over a period from 1 minute to 250 hours at a temperature from 0° C. to 150° C.

Preferably, the reactions are conducted in aromatic hydrocarbons, aliphatic hydrocarbons, ethers or mixtures thereof.

Preferably, the reaction is conducted in a solvent selected from among toluene, n-hexane, tetrahydrofuran, dioxane and diethyl ether.

The production of complexes of ruthenium 1 from complexes of ruthenium 2 and carbenes produced from precursor carbenes 6 is shown in Scheme II:

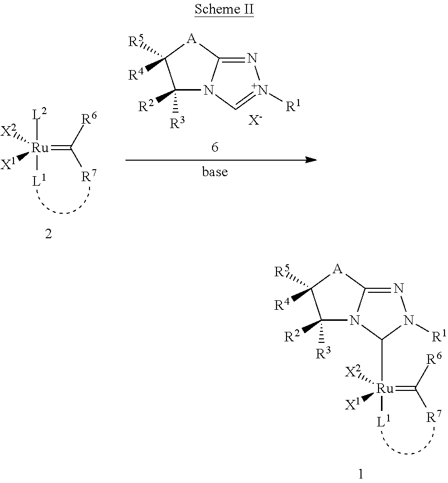

Preferably, the reaction is conducted over a period from 1 minute to 250 hours at a temperature from 0° C. to 150° C.

Preferably, the reaction is conducted in a protic or aprotic solvent, a chlorinated solvent or in a aromatic hydrocarbon solvent, or in mixtures thereof.

Preferably, the reaction is conducted in a solvent selected from among tetrahydrofuran and/or toluene and/or methylene chloride.

Preferably, the reaction is conducted in the presence of organic or inorganic bases.

Preferably, the reaction is conducted in the presence of bases selected from among: potassium tert-butanolate, potassium tert-amylate, potassium N,N-bis(trimethylsilyl)amide, sodium hydride.

The subject of the present invention is also the use of complexes of ruthenium defined by Formula 1 as (pre)catalysts in metathesis reactions and cycloisomerisation of olefins.

Preferably, ruthenium complexes defined by Formula 1 are used as (pre)catalysts in asymmetric ring closing metathesis (ARCM), in asymmetric ring opening metathesis with subsequent cross metathesis (AROM/CM) as well as in asymmetric cross metathesis (ACM).

The term “halogen atom” denotes an element selected from among F, Cl, Br and I.

The term “halide anion” denotes a fluoride, chloride, bromide or iodide anion.

The term “carbene” denotes a molecule containing a neutral carbon atom with the valence number 2 and two unpaired valence electrons. The term “carbene” also encompasses carbene analogues in which the carbon atom has been substituted by another chemical element such as boron, silicon, germanium, tin, lead, nitrogen, phosphorus, sulphur, selenium and tellurium.

The term “alkyl” refers to a saturated, linear or branched hydrocarbon substituent with an indicated number of carbon atoms. Examples of alkyl substituents are -methyl, -ethyl, -n-propyl, -n-butyl, -n-pentyl, -n-hexyl, -n-heptyl, -n-octyl, -n-nonyl, and -n-decyl.

Representative branched —(C₁-C₁₀) alkyls encompass -isopropyl, -sec-butyl, -isobutyl, -tert-butyl, -isopentyl, -neopentyl, -1-methylbutyl, -2-methylbutyl, -3-methylbutyl, -1,1-dimethylpropyl, -1,2-dimethylpropyl, -1-methylpentyl, -2-methylpentyl, -3-methylpentyl, -4-methylpentyl, -1-ethylbutyl, -2-ethylbutyl, -3-ethylbutyl, -1,1-dimethylbutyl, -1,2-dimethylbutyl, -1,3-dimethylbutyl, -2,2-dimethylbutyl, -2,3-dimethylbutyl, -3,3-dimethylbutyl, -1-methylhexyl, -2-methylhexyl, -3-methylhexyl, -4-methylhexyl, -5-methylhexyl, -1,2-dimethylpentyl, -1,3-dimethylpentyl, -1,2-dimethylhexyl, -1,3-dimethylhexyl, -3,3-dimethylhexyl, -1,2-dimethylheptyl, -1,3-dimethylheptyl, and -3,3-dimethylheptyl and the like.

The term “perfluoroalkyl” denotes an alkyl group as defined above, wherein all hydrogen atoms have been substituted by identical or different halide atoms.

The term “cycloalkyl” refers to a saturated mono- or polycyclic hydrocarbon substituent with the indicated number of carbon atoms.

Examples cycloalkyl substituents include -cyclopropyl; -cyclobutyl, -cyclopentyl, -cyclohexyl, -cycloheptyl, -cyclooctyl, -cyclononyl, -cyclfromecyl, and the like.

The term “alkoxyl” refers to alkyl or cycloalkyl substituents as defined above and attached via an oxygen atom.

The term “alkenyl” refers to an unsaturated, linear, or branched acyclic hydrocarbon substituent with the indicated number of carbon atoms and containing at least one double carbon-carbon bond. Examples of an alkenyl substituent include -vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, -1-hexenyl, -2-hexenyl, -3-hexenyl, -1-heptenyl, -2-heptenyl, -3-heptenyl, -1-octenyl, -2-octenyl, -3-octenyl, -1-nonenyl, -2-nonenyl, -3-nonenyl, -1-decenyl, -2-decenyl, -3-decenyl and the like.

The term “cycloalkenyl” refers to an unsaturated mono- or polycyclic hydrocarbon substituent with the indicated number of carbon atoms and containing at least one double carbon-carbon bond. Examples of a cycloalkenyl substituent include -cyclopentenyl, -cyclopentadienyl, -cyclohexenyl, -cyclohexadienyl, -cycloheptenyl, -cycloheptadienyl, -cycloheptatrienyl, -cyclooctenyl, -cyclooctadienyl, -cyclooctatrienyl, -cyclooctatetraenyl, -cyclononenyl, -cyclononadienyl, -cyclodecenyl, -cyclodecadienyl and the like.

The term “alkynyl” refers to an unsaturated, linear, or branched acyclic hydrocarbon substituent with the indicated number of carbon atoms and containing at least one triple carbon-carbon bond.

Examples of an alkynyl substituent include -acetylenyl, -propynyl, -1-butynyl, -2-butynyl, -1-pentynyl, -2-pentynyl, -3-methyl-1-butynyl, -4-pentynyl, -1-hexynyl, -2-hexynyl, -5-hexynyl and the like.

The term “cycloalkynyl” refers to an unsaturated mono- or polycyclic hydrocarbon substituent with the indicated number of carbon atoms and containing at least one triple carbon-carbon bond.

Examples of a cycloalkynyl substituent include -cyclohexynyl, -cycloheptynyl, -cyclooctynyl, and the like.

The term “aryl” refers to an aromatic mono- or polycyclic hydrocarbon substituent with the indicated number of carbon atoms, possibly substituted with at least one alkyl, alkoxyl, aryloxyl, a halogen atom, hydroxyl group, nitro group, ester group or ketone group, cyanide group, an amide, carboxyl group, sulfonamide group, formyl group or ether group. Examples of an aryl substituent include -phenyl, -tolyl, -xylyl, -naphthyl, -2,4,6-trimethylphenyl, -2-fluorophenyl, -4-fluorophenyl, -2,4,6-trifluorophenyl, -2,6-difluoro-4-nitrophenyl and the like.

The term “aralkyl” refers to alkyl substituents as defined above, substituted with at least one aryl as defined above. Examples of an aralkyl substituent include -benzyl, -diphenylmethyl, -triphenylmethyl and the like.

The term “heteroaryl” refers to an aromatic mono- or polycyclic hydrocarbon substituent with the indicated number of carbon atoms, in which at least one carbon atom has been substituted by a heteroatom selected from among O, N and S. Examples of heteroaryl substituents include -furyl, -thienyl, -imidazolyl, -oxazolyl, -thiazolyl, -isoxazolyl, -triazolyl, -oxadiazolyl, -tiadiazolyl, -tetrazolyl, -pyridyl, -pyrimidyl, -triazynyl, -indolyl, -benzo[b]furyl, -benzo[b]tienyl, -indazolyl, -benzoimidazolyl, -azaindolyl, -quinolyl, -isoquinolyl, -carbazolyl and the like.

The term “aryloxyl” refers to an aryl substituent as defined above, connected via an oxygen atom.

The term “heteroaryloxyl” refers to a heteroacyl substituent as defined above connected via an oxygen atom.

The term “heterocycle” refers to a saturated or partially unsaturated, mono- or polycyclic hydrocarbon substituent, with the indicated number of carbon atoms, in which at least one carbon atom has been substituted by a heteroatom selected from among O, N and S.

Examples of a heterocyclic substituent include -furyl, -thiophenyl, -pyrolyl, -oxazolyl, -imidazolyl, -thiazolyl, -isoxazolyl, -pirazolyl, -isothiazolyl, -triazynyl, -pyrolidynonyl, -pyrolidynyl, -hydantoinyl, -oxiranyl, -oxethanyl, -tetrahydrofuranyl, -tetrahydrotiophenyl, -quinolinyl, -isoquinolinyl, -chromonyl, -cumarynyl, -indolyl, -indolizynyl, -benzo [b]furanyl, -benzo[b]tiophenyl, -indazolyl, -purynyl, -4H-quinolizynyl, -isoquinolyl, -quinolyl, -phthalazynyl, -naphthyrydynyl, -carbazolyl, -/3-carbolinyl and the like.

The term “perfluoroaryl” denotes an aryl group as defined above in which all hydrogen atoms have been substituted by identical or different halogen atoms.

The term “neutral ligand” refers to a substituent devoid of charge, capable of coordinating with a metallic centre (ruthenium atom). Examples of such ligands may include: amines, phosphines and their oxides, phosphorines and alkyl and aryl phosphates, arsines and their oxides, ethers, aryl and alkyl sulphides, coordinated hydrocarbons, and alkyl and aryl halides.

The term “anionic ligand” refers to a substituent capable of coordinating with a metallic centre (ruthenium atom) possessing a charge, capable of partially or completely compensating the charge of the metallic centre. Examples of such ligands may be: such anions as fluoride, bromide, iodide, cyanide, cyanate and thiocyanate, carboxylic acid anions, alcohol anions, phenolic anions, thiol and thiophenol anions, hydrocarobon anions with a delocalised charge (i.e. cyclopentadiene), anions of (organo)sulphuric and (organo)phosphoric acids and esters thereof (such as i.e. anions of alkylsulphonic and arylsulphonic acids, anions of arylphsophoric and alkylphosphoric acids, anions of aryl and alkyl esters of sulphuric acids, anions of aryl and alkyl esters of phosphoric acids, anions of anions of aryl and alkyl esters of alkylphosphoric and arylophosphoric acids). Possibly, an anionic ligand may posses bound groups L₁ and L₂ such as a catechol anion, an acetylacetone anion or a salicyl aldehyde anion. The anionic ligands (X₁, X₂) and neutral ligands (L₁, L₂) may together form be may be connected to one another forming multidentate ligands, such as a: bidentate ligand (X₁, X₂), tridentate ligand (X₁, X₂, L₁), tetradentate ligand (X₁, X₂, L₁, L₂), bidentate ligand (X₁, L₁), tridentate ligand (X₁, L₁, L₂), or bidentate ligand (L₁, L₂). Examples of such ligands include a catechol anion, an acetylacetone anion as well as a salicylic aldehyde anion.

The examples below demonstrate the production and uses of the novel Complex 1.

### EXAMPLE I

### The Synthesis of Catalysts Defined by Formula 1d (According to Scheme I)

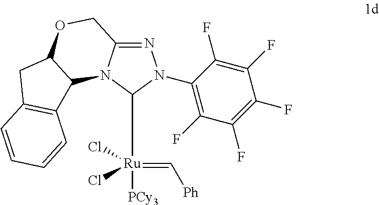

Using a protective atmosphere of argon, a Schlenk vessel was loaded with a carbene defined by Formula 3a (75.9 mg, 0.20 mmol)

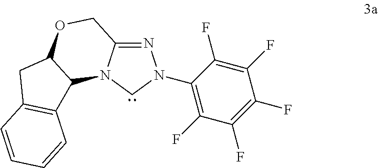

and dry deoxygenated toluene (4 mL) was added followed by solid carbene complex of ruthenium defined by Formula 2, in which X¹ and X² denote chlorine, L¹ and L² denotes tricyclohexylphosphine (PCy₃), R⁶ a hydrogen and R⁷ a phenyl (so-called 1st generation Grubbs catalyst, 164.6 mg, 0.20 mmol). The resulting solution was stirred at room temperature for 1 hour. From that moment onward, all subsequent operations were performed in the open air, without the need of a protective argon atmosphere. The reaction mixture was concentrated in an evaporator and loaded onto a chromatography column filled with a silica gel. The column was developed using an ethyl acetate-cyclohexane (10% v/v), and the brown fraction was collected. After evaporating off the solvent, we obtained Complex 1d in the form of a brown, microcrystalline solid (112 mg, 61% yield).

MS (FD/FI) calculated for C₄₃H₄₉Cl₂F₅N₃OPRu: 921.2; found: 921.2;

¹H NMR (CD₂Cl₂, 600 MHz)=19.80 (s), 7.67-7.61 (m), 7.38-7.30 (m), 7.30-7.15 (m), 5.10-4.60 (m), 3.40-2.70 (m), 2.40-2.30 (m), 1.85-1.35 (m), 1.16-1.00 (m) ppm.

¹³C NMR (CD₂Cl₂, 150 MHz)=152.6, 129.4, 127.8, 126.8, 33.0 (m), 30.4, 28.3 (m), 26.8 (m) ppm.

### EXAMPLE II

### The Synthesis of Catalysts Defined by Formula 1d (According to Scheme I)

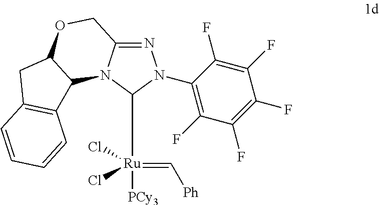

Using a protective atmosphere of argon, a Schlenk vessel was loaded with a complex of silver defined by Formula 4a (114.4 mg, 0.12 mmol), in which R¹ denotes pentafluorophenyl

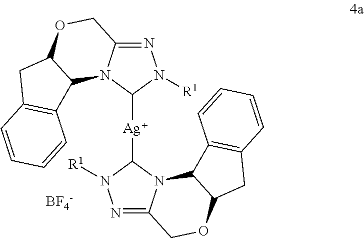

and dry deoxygenated toluene (4 mL) was added followed by solid carbene complexes of ruthenium defined by Formula 2, in which X¹ and X² denote chlorine, L¹ and L² denotes tricyclohexylphosphine (PCy₃), R⁶ a hydrogen and R⁷ a phenyl (so-called Ist generation Grubbs catalyst, 164.6 mg, 0.20 mmol). The resulting solution was stirred at room temperature for 1 hour. From that moment onward, all subsequent operations were performed in the open air, without the need of a protective argon atmosphere. The reaction mixture was concentrated in an evaporator and loaded onto a chromatography column filled with a silica gel. The column was developed using an ethyl acetate-cyclohexane (10% v/v), and the brown fraction was collected. After evaporating off the solvent, we obtained Complex 1d in the form of a brown, microcrystalline solid (108 mg, 59% yield).

### EXAMPLE III

### The Synthesis of Catalysts Defined by Formula 1d (According to Scheme II)

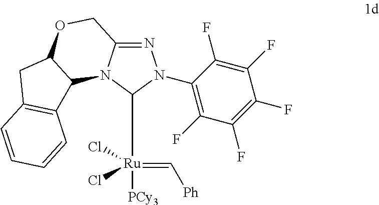

Using a protective atmosphere of argon, a Schlenk vessel was loaded with a carbene precursor defined by Formula 6a (93.4 mg, 0.20 mmol)

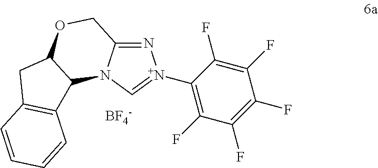

and dry deoxygenated toluene (4 mL) was added followed by potassium bis(trimethylsilyl)amide in toluene (0.5 M, 0.4 mL, 0.20 mmol). The resulting solution was stirred at room temperature for 15 minutes. Next, solid carbene complex of ruthenium defined by Formula 2, in which X¹ and X² denote chlorine, L¹ and L² denotes tricyclohexylphosphine (PCy₃), R⁶ a hydrogen and R⁷ a phenyl (so-called 1st generation Grubbs catalyst, 164.6 mg, 0.20 mmol) was added. The resulting solution was stirred at room temperature for 1 hour. From that moment onward, all subsequent operations were performed in the open air, without the need of a protective argon atmosphere. The reaction mixture was concentrated in an evaporator and loaded onto a chromatography column filled with a silica gel. The column was developed using an ethyl acetate-cyclohexane (10% v/v), and the brown fraction was colleted. After evaporating off the solvent, we obtained Complex 1d in the form of a brown, microcrystalline solid (87 mg, 47% yield).

### EXAMPLE IV

### The Synthesis of Catalysts Defined by Formula 1e (According to Scheme II)

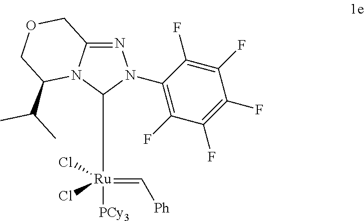

Using a protective atmosphere of argon, a Schlenk vessel was loaded with a carbene precursor defined by Formula 6b (84.2 mg, 0.20 mmol)

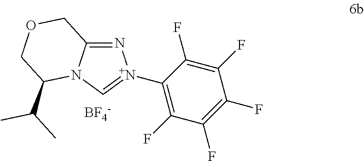

and dry deoxygenated toluene (4 mL) was added followed by potassium bis(trimethylsilyl)amide in toluene (0.5 M, 0.4 mL, 0.20 mmol). The resulting solution was stirred at room temperature for 15 minutes. Next, solid carbene complex of ruthenium defined by Formula 2, in which X¹ and X² denote chlorine, L¹ and L² denotes tricyclohexylphosphine (PCy₃), R⁶ a hydrogen and R⁷ phenyl (so-called 1st generation Grubbs catalyst, 164.6 mg, 0.20 mmol) was added. The resulting solution was stirred at room temperature for 1 hour. From that moment onward, all subsequent operations were performed in the open air, without the need of a protective argon atmosphere. The reaction mixture was concentrated in an evaporator and loaded onto a chromatography column filled with a silica gel. The column was developed using an ethyl acetate-cyclohexane (10% v/v), and the brown fraction was collected. After evaporating off the solvent, we obtained Complex 1e in the form of a brown, microcrystalline solid (85 mg, 49% yield).

MS (FD/FI) calculated for C₃₉H₅₁Cl₂F₅N₃OPRu: 875.2; found: 875.2;

¹H NMR (toluen-d8, 400 MHz)=20.39 (s), 7.30-7.14 (m), 4.46-4.28 (m), 4.01-3.89 (m), 3.80-3.72 (m), 3.59-3.52 (m), 3.22-3.12 (m), 2.90-2.75 (m), 2.50-2.30 (m), 2.05-1.80 (m), 1.80-1.10 (m), 0.87-0.77 (m) ppm.

### EXAMPLE V

### The Synthesis of Catalysts Defined by Formula 1f (According to Scheme I)

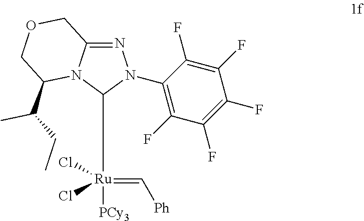

Using a protective atmosphere of argon, a Schlenk vessel was loaded with a complex of silver defined by Formula 4b (106.7 mg, 0.12 mmol), in which R¹ denotes pentafluorophenyl

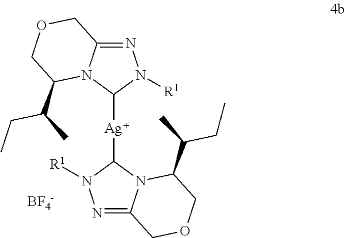

and dry deoxygenated tetrahydrofuran (4 mL) was added. Next, solid carbene complex of ruthenium defined by Formula 2, in which X¹ and X² denote chlorine, L¹ and L² denotes tricyclohexylphosphine (PCy₃), R⁶ a hydrogen and le phenyl (so-called 1st generation Grubbs catalyst, 164.6 mg, 0.20 mmol) was added. The resulting solution was stirred at room temperature for 1 hour. From that moment onward, all subsequent operations were performed in the open air, without the need of a protective argon atmosphere. The reaction mixture was concentrated in an evaporator and loaded onto a chromatography column filled with a silica gel. The column was developed using an ethyl acetate-cyclohexane (10% v/v), and the brown fraction was collected. After evaporating off the solvent, we obtained Complex 1f in the form of a brown, microcrystalline solid (102 mg, 57% yield).

MS (FD/FI) calculated for C₄₀H₅₃Cl₂F₅N₃OPRu: 889.2; found: 889.2;

¹H NMR (benzen-d6, 600 MHz)=20.34 (s), 8.72 (d, J=7.6), 8.33-8.28 (m) 6.99-6.94 (m), 4.45 (d, J=15.9), 4.00 (d, J=15.9), 2.46-2.34 (m), 2.02-1.83 (m), 1.76-1.52 (m), 1.20-1.06 (m) ppm.

¹³C NMR (benzen-d6, 150 MHz)=309.3, 189.0, 153.7, 153.0, 152.7, 150.6, 150.5, 131.8, 131.6, 130.7, 130.6, 129.7, 129.6, 128.7, 64.8, 64.7, 61.9, 61.8, 58.7, 57.4, 40.3, 36.2, 35.8, 33.6 (d, J_{CP}=16), 32.8, 32.7, 32.6, 32.1, 32.0, 30.5, 30.3, 28.5, 28.4 (d, J_{CP}=10), 27.6, 27.5, 27.4, 27.3, 27.2, 27.1, 26.9, 14.9, 14.5, 12.5, 12.2 ppm.

### EXAMPLE VI

### The Use of Complex 1 as (Pre)Catalysts for Asymmetric Ring Opening Metathesis with a Cross Metathesis (AROM/CM) of Compound S1

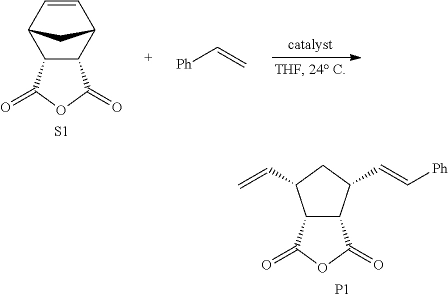

Using a protective atmosphere of argon, a Schlenk vessel was loaded with the substrate S1 (32.8 mg, 0.2 mmol), styrene (41.7 mg, 0.4 mmol, 2 eq.) and dry deoxygenated tetrahydrofuran (1 mL). Next, solid carbene complex of ruthenium defined by Formula if (0.004 mmol, 2 mol %) was added. The mixture was stirred at a temperature of 24° C. for 24 hours. After this time ethyl-vinyl ether (0.5 mL) was added and after 30 minutes mixture was evaporated. The product P1 was isolated using column chromatography on a silica gel (ethyl acetate/cyclohexane=1:4 v/v). The product was analyzed using high performance liquid chromatography with a chiral column (Chiralcel® OJ, n-hexane/isopropanol=1:1 v/v; 0.7 ml/min; 254 nm). Colourless solid (40 mg, 75%), ee=67%.

### EXAMPLE VII

### The Use of Complex 1 as a (Pre)Catalyst for Asymmetric Ring Opening Metathesis with a Cross Metathesis (AROM/CM) of Compound S2

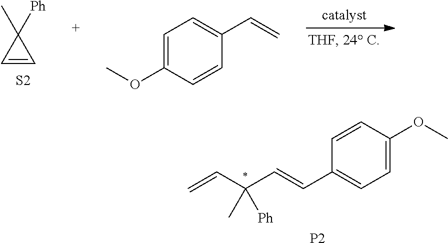

Using a protective atmosphere of argon, a Schlenk vessel was loaded with the substrate S2 (13.0 mg, 0.1 mmol), 4-vinyloanizol (26.8 mg, 0.2 mmol, 2 eq.) and dry deoxygenated tetrahydrofuran (0.5 mL). Next, solid carbene complex of ruthenium defined by Formula 1e (0.005 mmol, 5 mol %) was added. The mixture was stirred at a temperature 24° C. for 24 hours. After this time ethyl-vinyl ether (0.5 mL) was added and after 30 minutes mixture was evaporated. The product P2 was isolated using column chromatography on a silica gel (ethyl acetate/cyclohexane=5:95 v/v). The product was analyzed using high performance liquid chromatography with chiral column (Chiralcel® OD-H, n-hexane; 1.0 ml/min; 254 nm). Colourless oil (21 mg, 79%), ee=48%.

1. A ruthenium complex defined by Formula 1 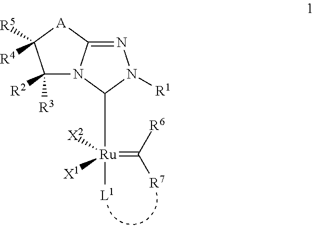
in which:
R¹ denotes a C₅-C₂₄ perfluoroaryl;
R², R³, R⁴ and R⁵ independently of one another denote a hydrogen atom, a halogen atom, a C₁-C₂₅ alkyl, a C₃-C₇ cycloalkyl, a alkoxyl C₁-C₂₅, aryloxyl C₅-C₂₄, heteroaryloxyl C₅-C₂₀, a C₅-C₂₄ aryl, a C₅-C₂₀ heteroaryl, a C₇-C₂₄ aralkyl, a C₅-C₂₄ perfluoroaryl, a 3-12 membered heterocycle, wherein the groups R², R³, R⁴ and R⁵ may be mutually connected into a ring;
A denotes a —CH₂—, —O— or —OCH₂— group;
R⁶ and R⁷ independently of one another denote a hydrogen atom, a halogen atom, a C₁-C₂₅ alkyl, a C₁-C₂₅ perfluoroalkyl, a C₂-C₂₅ alkene, a C₃-C₇ cycloalkyl, a C₂-C₂₅ alkenyl, cycloalkenyl C₃-C₂₅, alkynyl C₂-C₂₅, cycloalkynyl C₃-C₂₅, a alkoxyl C₁-C₂₅, aryloxyl C₅-C₂₄, heteroaryloxyl C₅-C₂₀, a C₅-C₂₄ aryl, a C₅-C₂₀ heteroaryl, a C₇-C₂₄ aralkyl, a C₅-C₂₄ perfluoroaryl, or a 3-12 membered heterocycle wherein the alkyl groups may be mutually connected into a ring, wherein R⁶ and R⁷ preferably denote a hydrogen, aryl substituted with a nitro (—NO₂), cyanide (—CN), carboxyl (—COOH), ester (—COOR′), amide (—CONR′₂), sulfonyl (—SO₂R′), formyl (—CHO), sulfonamide (—SO₂NR′₂) or ketone (—COR′) group, in which R′ has the following meaning: a C₁-C₅ alkyl, a C₁-C₅ perfluoroalkyl, a C₅-C₂₄ aryl, a C₇-C₂₄ aralkyl, a C₅-C₂₄ perfluoroaryl;
L¹ denotes a neutral ligand selected from groups encompassing pyridine or substituted pyridine, P(R′)₃, P(OR′)₃, O(R′)₂, N(R′)₃, where each R′ independently denotes a C₁-C₁₂ alkyl, a C₃-C₁₂ cycloalkyl, a C₅-C₂₀ aryl, a C₇-C₂₄ aralkyl, a C₅-C₂₄ perfluoroaryl, or a 5-12 membered heteroaryl;
X¹ and X² denote an anionic ligand independently selected from groups encompassing halide anions, the groups —CN, —SCN, —OR′, —SR′, —O(C═O)R′, —O(SO₂)R′, and —OSi(R′)₃, where R′ denotes a C₁-C₁₂ alkyl, a C₃-C₁₂ cycloalkyl, a C₂-C₁₂ alkenyl, or a C₅-C₂₀ aryl, which may possibly be substituted with at least one C₁-C₁₂ alkyl, a C₁-C₁₂ perfluoroalkyl, a C₁-C₁₂ alkoxyl, a C₅-C₂₄ aryloxyl, a C₅-C₂₀ heteroaryloxyl or a halogen atom.

2. A complex according to claim 1, characterised in that it is a compound defined by Formula 1a 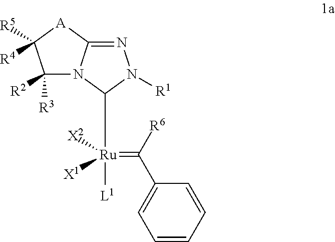
in which:
R¹, R², R³, R⁴, R⁵, A, L¹, X¹ and X² have the same meaning as in Formula 1;
R⁶ denotes a hydrogen.

3. A complex according to claim 1, characterised in that it is a compound defined by Formula 1b 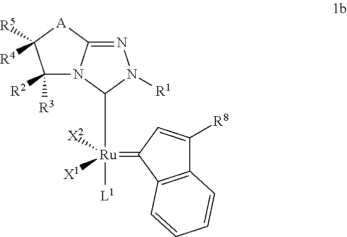
in which:
R¹, R², R³, R⁴, R⁵, A, L¹, X¹ and X² have the same meaning as in Formula 1;
R⁸ denotes a hydrogen atom, a C₅-C₂₀ aryl, a C₅-C₂₀ heteroaryl, a C₇-C₂₄ aralkyl, vinyl or allenyl.

4. A complex according to claim 1, characterised in that it is a compound defined by Formula 1c 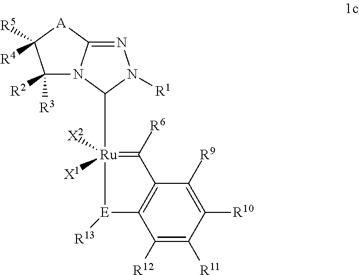
in which:
R¹, R², R³, R⁴, R⁵, A, L¹, X¹ and X² have the same meaning as in Formula 1
R⁶ denotes a hydrogen
R⁹, R¹⁰, R¹¹, R¹² independently of one another denote a hydrogen atom, a halogen atom, a C₁-C₂₅ alkyl, a C₁-C₂₅ perfluoroalkyl, a C₂-C₂₅ alkene, a C₃-C₇ cycloalkyl, a C₂-C₂₅ alkenyl, cycloalkenyl C₃-C₂₅, alkynyl C₂-C₂₅, cycloalkynyl C₃-C₂₅, a C₅-C₂₄ aryl, a C₅-C₂₀ heteroaryl, a C₇-C₂₄ aralkyl, a C₅-C₂₄ perfluoroaryl, a 3-12 membered heterocycle wherein the alkyl groups may be mutually connected into a ring, an ether (—OR′), thioether (—SR′), nitro (—NO₂), cyanide (—CN), carboxyl (—COOH), ester (—COOR′), amide (—CONR′₂), imide (—CONR′COR′), amino (—NR′₂), amide (NR′COR′), sulfonamide (—NR′SO₂R′), sulfonyl (—SO₂R′), formyl (—CHO), sulfonamide (—SO₂NR′₂), or ketone (—COR′) group, in which R′ has the following meaning: a C₁-C₅ alkyl, a C₁-C₅ perfluoroalkyl, a C₅-C₂₄ aryl, a C₅-C₂₄ perfluoroaryl, a C₇-C₂₄ aralkyl, wherein the alkyl groups may be mutually connected into a ring, wherein R⁹, R¹⁰, R₁₁, R¹² preferably denotes a hydrogen;
R¹³ denotes a hydrogen atom, a C₁-C₂₅ alkyl, a C₁-C₂₅ perfluoroalkyl, a C₃-C₇ cycloalkyl, a C₅-C₂₄ aryl, a C₅-C₂₄ perfluoroaryl, a C₅-C₂₀ heteroaryl, a C₇-C₂₄ aralkyl, a 3-12 membered heterocycle wherein the alkyl groups may be mutually connected into a ring, a —COR′ acyl, cyanide (—CN), carboxyl (—COOH), ester (—COOR′), amide (—CONR′₂), sulfonyl (—SO₂R′), formyl (—CHO), sulfonamide (—SO₂NR′₂), or ketone (—COR′) group, in which R′ has the following meaning: a C₁-C₅ alkyl, a C₁-C₅ perfluoroalkyl, a C₅-C₂₄ aryl, a C₅-C₂₄ perfluoroaryl, a C₇-C₂₄ aralkyl;
E denotes an oxygen atom.

5. A complex according to claim 1, characterised in that
R¹ denotes pentafluorophenyl;
R², R³, R⁴ and R⁵ independently of one another denote a hydrogen atom, a C₁-C₂₅ alkyl, a C₃-C₇ cycloalkyl, a C₅-C₂₄ aryl, a C₅-C₂₀ heteroaryl, a C₇-C₂₄ aralkyl, a C₅-C₂₄ perfluoroaryl, a 3-12 membered heterocycle, wherein the groups R², R³, R⁴ and R⁵ may be mutually connected into a ring;
A denotes a —CH₂—, —O— or —OCH₂— group;
L¹ denotes a neutral ligand selected from groups encompassing tricyclohexylphosphine, triphenylphosphine, pyridine, 3-bromopyridine;
X¹ and X² denote chlorine, bromine or iodine.

6. A method of producing a ruthenium complex defined in claim 1, characterised in that the carbene ruthenium complex defined by Formula 2 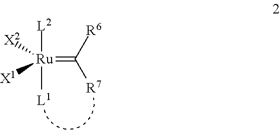
in which:
R⁶ and R⁷ independently of one another denote a hydrogen atom, a halogen atom, a C₁-C₂₅ alkyl, a C₁-C₂₅ perfluoroalkyl, a C₂-C₂₅ alkene, a C₃-C₇ cycloalkyl, a C₂-C₂₅ alkenyl, cycloalkenyl C₃-C₂₅, alkynyl C₂-C₂₅, cycloalkynyl C₃-C₂₅, a alkoxyl C₁-C₂₅, aryloxyl C₅-C₂₄, heteroaryloxyl C₅-C₂₀, a C₅-C₂₄ aryl, a C₅-C₂₀ heteroaryl, a C₇-C₂₄ aralkyl, a C₅-C₂₄ perfluoroaryl, a 3-12 membered heterocycle wherein the alkyl groups may be mutually connected into a ring, wherein R⁶ and R⁷ preferably denote a hydrogen, aryl substituted with a nitro (—NO₂), cyanide (—CN), carboxyl (—COOH), ester (—COOR′), amide (—CONR′₂), sulfonyl (—SO₂R′), formyl (—CHO), sulfonamide (—SO₂NR′₂) or ketone (—COR′) group, in which R′ has the following meaning: a C₁-C₅ alkyl, a C₁-C₅ perfluoroalkyl, a C₅-C₂₄ aryl, a C₇-C₂₄ aralkyl, a C₅-C₂₄ perfluoroaryl,
L¹ and L² denote a neutral ligand selected from groups encompassing pyridine or substituted pyridine, P(R′)₃, P(OR′)₃, O(R′)₂, N(R′)₃, where each R′ independently denotes a C₁-C₁₂ alkyl, a C₃-C₁₂ cycloalkyl, a C₅-C₂₀ aryl, a C₇-C₂₄ aralkyl, a C₅-C₂₄ perfluoroaryl, or a 5-12 membered heteroaryl,
X¹ and X² denote an anionic ligand independently selected from groups encompassing halide anions, a —CN, —SCN, —OR′, —SR′, —O(C═O)R′, —O(SO₂)R′ or —OSi(R′)₃ group, where R′ denotes a C₁-C₁₂ alkyl, a C₃-C₁₂ cycloalkyl, a C₂-C₁₂ alkenyl, or a C₅-C₂₀ aryl, which may possibly be substituted with at least one C₁-C₁₂ alkyl, a C₁-C₁₂ perfluoroalkyl, a C₁-C₁₂ alkoxyl, a C₅-C₂₄ aryloxyl, a C₅-C₂₀ heteroaryloxyl or a halogen atom,
is subjected to a reaction with a chiral, non-racemic carbene defined by Formula 3 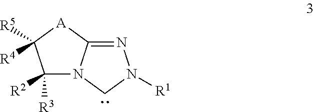
in which:
R¹ denotes a C₅-C₂₄ perfluoroaryl;
R², R³, R⁴ and R⁵ independently of one another denote a hydrogen atom, a halogen atom, a C₁-C₂₅ alkyl, a C₃-C₇ cycloalkyl, a alkoxyl C₁-C₂₅, aryloxyl C₅-C₂₄, heteroaryloxyl C₅-C₂₀, a C₅-C₂₄ aryl, a C₅-C₂₀ heteroaryl, a C₇-C₂₄ aralkyl, a C₅-C₂₄ perfluoroaryl, a 3-12 membered heterocycle, wherein the groups R², R³, R⁴ and R⁵ may be mutually connected into a ring;
A denotes a —CH₂—, —O— or —OCH₂— group.

7. A method of producing a ruthenium complex defined in claim 1, characterised in that the carbene ruthenium complex defined by Formula 2 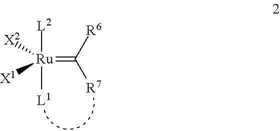
in which:
R⁶ and R⁷ independently of one another denote a hydrogen atom, a halogen atom, a C₁-C₂₅ alkyl, a C₁-C₂₅ perfluoroalkyl, a C₂-C₂₅ alkene, a C₃-C₇ cycloalkyl, a C₂-C₂₅ alkenyl, cycloalkenyl C₃-C₂₅, alkynyl C₂-C₂₅, cycloalkynyl C₃-C₂₅, a alkoxyl C₁-C₂₅, aryloxyl C₅-C₂₄, heteroaryloxyl C₅-C₂₀, a C₅-C₂₄ aryl, a C₅-C₂₀ heteroaryl, a C₇-C₂₄ aralkyl, a C₅-C₂₄ perfluoroaryl, a 3-12 membered heterocycle wherein the alkyl groups may be mutually connected into a ring, wherein R⁶ and R⁷ preferably denote a hydrogen, aryl substituted with a nitro (—NO₂), cyanide (—CN), carboxyl (—COOH), ester (—COOR′), amide (—CONR′₂), sulfonyl (—SO₂R′), formyl (—CHO), sulfonamide (—SO₂NR′₂) or ketone (—COR′) group, in which R′ has the following meaning: a C₁-C₅ alkyl, a C₁-C₅ perfluoroalkyl, a C₅-C₂₄ aryl, a C₇-C₂₄ aralkyl, a C₅-C₂₄ perfluoroaryl,
L¹ and L² denote a neutral ligand selected from groups encompassing pyridine or substituted pyridine, P(R′)₃, P(OR′)₃, O(R′)₂, N(R′)₃, where each R′ independently denotes a C₁-C₁₂ alkyl, a C₃-C₁₂ cycloalkyl, a C₅-C₂₀ aryl, a C₇-C₂₄ aralkyl, a C₅-C₂₄ perfluoroaryl, or a 5-12 membered heteroaryl,
X¹ and X² denote an anionic ligand independently selected from groups encompassing halide anions, —CN, —SCN, —OR′, —SR′, —O(C═O)R′, —O(SO₂)R′, or —OSi(R′)₃ groups where R′ denotes a C₁-C₁₂ alkyl, a C₃-C₁₂ cycloalkyl, a C₂-C₁₂ alkenyl, or a C₅-C₂₀ aryl, which may possibly be substituted with at least one C₁-C₁₂ alkyl, a C₁-C₁₂ perfluoroalkyl, a C₁-C₁₂ alkoxyl, a C₅-C₂₄ aryloxyl, a C₅-C₂₀ heteroaryloxyl or a halogen atom,
is subjected to a reaction with a chiral, non-racemic complex of silver defined by Formula 4 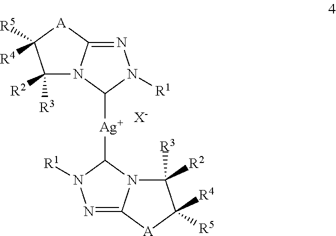
in which:
R¹ denotes C₅-C₂₄ perfluoroaryl;
R₂, R³, R⁴ and R⁵ independently of one another denote a hydrogen atom, a halogen atom, a C₁-C₂₅ alkyl, a C₃-C₇ cycloalkyl, a alkoxyl C₁-C₂₅, aryloxyl C₅-C₂₄, heteroaryloxyl C₅-C₂₀, a C₅-C₂₄ aryl, a C₅-C₂₀ heteroaryl, a C₇-C₂₄ aralkyl, a C₅-C₂₄ perfluoroaryl, a 3-12 membered heterocycle, wherein the groups R², R³, R⁴ and R⁵ may be mutually connected into a ring;
A denotes a —CH₂—, —O— or —OCH₂— group;
X denotes a halide anion or BF₄⁻, PF₆⁻ or ClO₄.

8. A method of producing a ruthenium complex defined in claim 1, characterised in that the carbene ruthenium complex defined by Formula 2 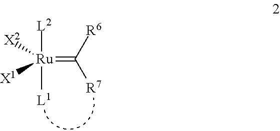
in which:
R⁶ and R⁷ independently of one another denote a hydrogen atom, a halogen atom, a C₁-C₂₅ alkyl, a C₁-C₂₅ perfluoroalkyl, a C₂-C₂₅ alkene, a C₃-C₇ cycloalkyl, a C₂-C₂₅ alkenyl, cycloalkenyl C₃-C₂₅, alkynyl C₂-C₂₅, cycloalkynyl C₃-C₂₅, a alkoxyl C₁-C₂₅, aryloxyl C₅-C₂₄, heteroaryloxyl C₅-C₂₀, a C₅-C₂₄ aryl, a C₅-C₂₀ heteroaryl, a C₇-C₂₄ aralkyl, a C₅-C₂₄ perfluoroaryl, a 3-12 membered heterocycle wherein the alkyl groups may be mutually connected into a ring, wherein R⁶ and R⁷ preferably denote a hydrogen, an aryl substituted with a nitro (—NO₂), cyanide (—CN), carboxyl (—COOH), ester (—COOR′), amide (—CONR′₂), sulfonyl (—SO₂R′), formyl (—CHO), sulfonamide (—SO₂NR′₂) or ketone (—COR′) group, in which R′ has the following meaning: a C₁-C₅ alkyl, a C₁-C₅ perfluoroalkyl, a C₅-C₂₄ aryl, a C₇-C₂₄ aralkyl, a C₅-C₂₄ perfluoroaryl,
L¹ and L² denote a neutral ligand selected from groups encompassing pyridine or substituted pyridine, P(R′)₃, P(OR′)₃, O(R′)₂, N(R′)₃, where each R′ independently denotes a C₁-C₁₂ alkyl, a C₃-C₁₂ cycloalkyl, a C₅-C₂₀ aryl, a C₇-C₂₄ aralkyl, a C₅-C₂₄ perfluoroaryl, or a 5-12 membered heteroaryl,
X¹ and X² denote an anionic ligand independently selected from groups encompassing halide anions, a —CN, —SCN, —OR′, —SR′, —O(C═O)R′, —O(SO₂)R, —OSi(R′)₃ group, where R′ denotes C₁-C₁₂ alkyl, a C₃-C₁₂ cycloalkyl, a C₂-C₁₂ alkenyl, or a C₅-C₂₀ aryl, which may possibly be substituted with at least one C₁-C₁₂ alkyl, a C₁-C₁₂ perfluoroalkyl, a C₁-C₁₂ alkoxyl, a C₅-C₂₄ aryloxyl, a C₅-C₂₀ heteroaryloxyl or a halogen atom,
is subjected to a reaction with a chiral, non racemic carbene precursor defined by Formula 5 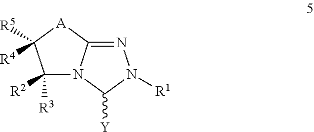
in which:
R¹ denotes C₅-C₂₄ perfluoroaryl;
R², R³, R⁴ and R⁵ independently of one another denote a hydrogen atom, a halogen atom, a C₁-C₂₅ alkyl, a C₃-C₇ cycloalkyl, a alkoxyl C₁-C₂₅, aryloxyl C₅-C₂₄, heteroaryloxyl C₅-C₂₀, a C₅-C₂₄ aryl, a C₅-C₂₀ heteroaryl, a C₇-C₂₄ aralkyl, a C₅-C₂₄ perfluoroaryl, a 3-12 membered heterocycle, wherein the groups R², R³, R⁴ and R⁵ may be mutually connected into a ring;
A denotes a —CH₂—, —O— or —OCH₂— group;
Y denotes an alkoxyl, pentafluorophenyl or —CCl₃.

9. A method of producing a ruthenium complex defined in claim 1, characterised in that the carbene ruthenium complex defined by Formula 2 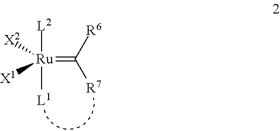
in which:
R⁶ and R⁷ independently of one another denote a hydrogen atom, a halogen atom, a C₁-C₂₅ alkyl, a C₁-C₂₅ perfluoroalkyl, a C₂-C₂₅ alkene, a C₃-C₇ cycloalkyl, a C₂-C₂₅ alkenyl, cycloalkenyl C₃-C₂₅, alkynyl C₂-C₂₅, cycloalkynyl C₃-C₂₅, a alkoxyl C₁-C₂₅, aryloxyl C₅-C₂₄, heteroaryloxyl C₅-C₂₀, a C₅-C₂₄ aryl, a C₅-C₂₀ heteroaryl, a C₇-C₂₄ aralkyl, a C₅-C₂₄ perfluoroaryl, a 3-12 membered heterocycle wherein the alkyl groups may be mutually connected into a ring, wherein R⁶ and R⁷ preferably denote a hydrogen, aryl substituted with a nitro (—NO₂), cyanide (—CN), carboxyl (—COOH), ester (—COOR′), amide (—CONR′₂), sulfonyl (—SO₂R′), formyl (—CHO), sulfonamide (—SO₂NR′₂) or ketone (—COR′) group, in which R′ has the following meaning: a C₁-C₅ alkyl, a C₁-C₅ perfluoroalkyl, a C₅-C₂₄ aryl, a C₇-C₂₄ aralkyl, a C₅-C₂₄ perfluoroaryl,
L¹ and L² denote a neutral ligand selected from groups encompassing pyridine or substituted pyridine, P(R′)₃, P(OR′)₃, O(R′)₂, N(R′)₃, where each R′ independently denotes a C₁-C₁₂ alkyl, a C₃-C₁₂ cycloalkyl, a C₅-C₂₀ aryl, a C₇-C₂₄ aralkyl, a C₅-C₂₄ perfluoroaryl, or a 5-12 membered heteroaryl,
X¹ and X² denote an anionic ligand independently selected from groups encompassing halide anions, a —CN, —SCN, —OR′, —SR′, —O(C═O)R, —O(SO₂)R′, —OSi(R′)₃, where R′ denotes C₁-C₁₂ alkyl, a C₃-C₁₂ cycloalkyl, a C₂-C₁₂ alkenyl, or a C₅-C₂₀ aryl, which may possibly be substituted with at least one C₁-C₁₂ alkyl, a C₁-C₁₂ perfluoroalkyl, a C₁-C₁₂ alkoxyl, a C₅-C₂₄ aryloxyl, a C₅-C₂₀ heteroaryloxyl or a halogen atom,
is subjected to a reaction with a carbene formed as a result of the reaction of potassium tert-amylate or potassium tert-butanolate or potassium N,N-bis(trimethylsilyl)amide or sodium hydride with a chiral, non-racemic carbene precursor defined by Formula 6 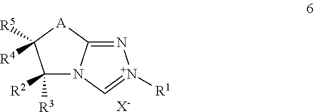
in which:
R¹ denotes C₅-C₂₄ perfluoroaryl;
R², R³, R⁴ and R⁵ independently of one another denote a hydrogen atom, a halogen atom, a C₁-C₂₅ alkyl, a C₃-C₇ cycloalkyl, a alkoxyl aryloxyl C₅-C₂₄, heteroaryloxyl C₅-C₂₀, a C₅-C₂₄ aryl, a C₅-C₂₀ heteroaryl, a C₇-C₂₄ aralkyl, a C₅-C₂₄ perfluoroaryl, a 3-12 membered heterocycle, wherein the groups R², R³, R⁴ and R⁵ may be mutually connected into a ring;
A denotes —CH₂—, —O— or —OCH₂— group;
X denotes a halide anion or BF₄⁻, PF₆⁻ or ClO₄⁻.

10. The method of producing a ruthenium complex according to claim 6, characterised in that as the carbene ruthenium complex use is made of a compound defined by Formula 2a 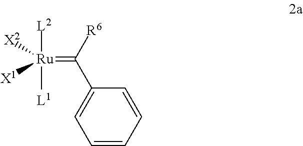
in which:
X¹, X², L¹ and L² have the same meaning as in Formula 2,
R⁶ denotes a hydrogen.

11. The method of producing a ruthenium complex according to claim 6, characterised in that as the carbene ruthenium complex use is made of a compound defined by Formula 2b 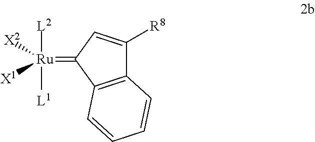
in which
X¹, X², L¹ and L² have the same meaning as in Formula 2,
R⁸ denotes a hydrogen atom, a C₅-C₂₀ aryl, a C₅-C₂₀ heteroaryl, a C₇-C₂₄ aralkyl, vinyl or allenyl.

12. The method of producing a ruthenium complex according to claim 6, characterised in that as the carbene ruthenium complex use is made of a compound defined by Formula 2c 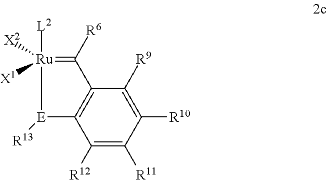
in which
X¹, X², L² have the same meaning as in Formula 2,
R⁶ denotes a hydrogen,
R⁹, R¹⁰, R¹¹, R¹² independently of one another denote a hydrogen atom, a halogen atom, a C₁-C₂₅ alkyl, a C₁-C₂₅ perfluoroalkyl, a C₂-C₂₅ alkene, a C₃-C₇ cycloalkyl, a C₂-C₂₅ alkenyl, cycloalkenyl C₃-C₂₅, alkynyl C₂-C₂₅, cycloalkynyl C₃-C₂₅, a C₅-C₂₄ aryl, a C₅-C₂₀ heteroaryl, a C₇-C₂₄ aralkyl, a C₅-C₂₄ perfluoroaryl, a 3-12 membered heterocycle wherein the alkyl groups may be mutually connected into a ring, an ether (—OR′), thioether (—SR′), nitro (—NO₂), cyanide (—CN), carboxyl (—COOH), ester (—COOR′), amide (—CONR′₂), imide (—CONR′COR′), amino (—NR′₂), amide (NR′COR′), sulfonamide (—NR′SO₂R′), sulfonyl (—SO₂R′), formyl (—CHO), sulfonamide (—SO₂NR′₂) or ketone (—COR′) group, in which R′ has the following meaning: a C₁-C₅ alkyl, a C₁-C₅ perfluoroalkyl, a C₅-C₂₄ aryl, a C₅-C₂₄ perfluoroaryl, a C₇-C₂₄ aralkyl, wherein the alkyl groups may be mutually connected into a ring, wherein R⁹, R¹⁰, R¹¹ and R¹² preferably denotes a hydrogen;
R¹³ denotes a hydrogen atom, a C₁-C₂₅ alkyl, a C₁-C₂₅ perfluoroalkyl, a C₃-C₇ cycloalkyl, a C₅-C₂₄ aryl, a C₅-C₂₄ perfluoroaryl, a C₅-C₂₀ heteroaryl, a C₇-C₂₄ aralkyl, a 3-12 membered heterocycle wherein the alkyl groups may be mutually connected into a ring, a —COR′ acyl group, cyanide (—CN), carboxyl (—COOH), ester (—COOR′), amide (—CONR′₂), sulfonyl (—SO₂R′), formyl (—CHO), sulfonamide (—SO₂NR′₂) or ketone (—COR′) group, in which R′ has the following meaning: a C₁-C₅ alkyl, a C₁-C₅ perfluoroalkyl, a C₅-C₂₄ aryl, a C₅-C₂₄ perfluoroaryl, a C₇-C₂₄ aralkyl, a C₅-C₂₄ perfluoroaryl;
E denotes an oxygen atom.

13. The method of producing a ruthenium complex according to claim 6, characterised in that the reaction is conducted over a period from 1 min to 250 hrs, at a temperature from 0 to 150° C.

14. The method of producing a ruthenium complex according to claim 6, characterised in that the reaction is conducted in a protic or aprotic solvent, a chlorinated solvent or in an aromatic hydrocarbon solvent, or in mixtures thereof.

15. The method of producing a ruthenium complex according to claim 6, characterised in that the reaction is conducted in a solvent selected from among tetrahydrofuran and/or toluene and/or methylene chloride.

16. A method for the metathesis and cycloisomerisation of olefins comprising adding the ruthenium complex defined by Formula 1 as defined in claim 1 to reactants utilized in the metathesis and cycloisomerisation of olefins.

17. The method according to claim 16, characterized in that the ruthenium complex is present as a (pre)catalyst in asymmetric ring opening metathesis with cross metathesis (AROM/CM), asymmetric cross metathesis (ACM), and asymmetric ring closure metathesis (ARCM).
